(19) United States
(12) Patent Application Publication
(10) Pub. No.: US 2007/0276427 A1
(43) Pub. Date: Nov. 29, 2007

(54) Torquable balloon catheters and methods

(76) Inventor: Charles Louis Euteneuer, St. Michael, MN (US)

Correspondence Address: CYR & ASSOCIATES, P.A., 605 U.S. Highway 169, Suite 300, Plymouth, MN, 55441, US

(21) Appl. No.: 11/442,089

(22) Filed: May 26, 2006

(51) Int. Cl. A61M 5/178; A61M 29/00

(52) U.S. Cl. 606/192; 604/164.13

(57) ABSTRACT

Torquable balloon apparatus to access bodily lumen of a patient are disclosed. The torquable balloon apparatus include an inflation tube, a torque tube a core wire and a balloon. The torque tube is secured over the core wire. The balloon is secured to a distal end of the inflation tube. The balloon is slidably secured over the portion of the torque tube extending from the distal end of the inflation tube. A proximal tube may be provided at the proximal end of the inflation tube.

### BACKGROUND OF THE INVENTION

1. Summary of the Invention

The present inventions relate to medical devices and, more particularly, to medical catheters and medical guidewires for insertion into bodily lumen of patients.

2. Description of the Related Art

Medical catheters and guidewires can be useful tools in treating intravascular disorders, disorders within other lumen of the body, extracting fluids from lumen as well as introducing fluid into lumen. Some medical catheters and most guidewires are configured to be received through a medical device to permit the medical device to be slid over the medical catheter or guidewire and positioned within the body of a patient. Further, many catheters and some guidewire designs include a balloon at or near the distal end of the catheter or guidewire. Depending on the configuration, these devices can also be used to introduce and/or expand various other medical devices, such as stents for example. The balloons may help direct the distal end of the catheter through a lumen where the pulsatile flow of blood may permit them to act as a “sail.” Further, the balloons in various configurations may be used to test for the occlusion of vessels, for embolization for bleeding, to treat or control vasospasms, and for treatment of nosebleeds, among other uses.

Medical catheters and guidewires are particularly useful in accessing remote and tortuous locations within the body. Because of the need to navigate through the body to remote locations through narrow twisting lumen, medical catheters and guidewires are frequently long thin devices. Frequently, the procedures using medical catheters and guidewires are time sensitive. Accordingly, these devices need to be easily guidable in an efficient manner by a user.

The medical catheters and guidewires frequently are subjected to various localized forces as they are torqued and pushed into position by a surgeon and the devices come into contact with various bodily structures within a patient. During positioning procedures, some configurations of the balloons positioned distally on the catheter or guidewire can collapse, bend, twist or otherwise deform. This twisting can form creases in the balloon that can catch or otherwise impede the navigation of the bodily lumen. This can inhibit or slow the surgeon's placement of the balloon at a desired location in the patient which in certain circumstances can be deleterious to a patient. The deformation of the balloon during implantation may also prevent or alter the balloons ability to be expanded after the surgeon has properly positioned the balloon which again can be deleterious to a patient. The deformation of the balloon after inflation can prevent or alter the balloons ability to be deflated as a surgeon is removing or preparing to remove the medical catheter and guidewire from the patient. Among other problems, the ability to deflate the balloon can complicate or prevent the ability to remove the medical catheter and guidewire from the patient. Accordingly, a need exists for balloon configurations that provide desired performance characteristics while being resistant to deformation during positioning procedures.

### SUMMARY OF THE INVENTION

Apparatus and methods in accordance with the present invention may resolve many of the needs and shortcomings discussed above and will provide additional improvements and advantages as will be recognized by those skilled in the art upon review of the present disclosure.

The present inventions provide torquable balloon apparatus for accessing a target location in a bodily lumen of a patient. A torquable balloon apparatus in accordance with one or more of the present inventions may include an inflation tube, a torque tube and a balloon. In one aspect, the torquable balloon apparatus may further include a proximal tube. The proximal tube may define a proximal lumen. The proximal lumen may extend between a proximal end and a distal end of the proximal tube. The distal end of the proximal tube may be secured to a proximal end of the inflation tube with the proximal lumen of the proximal tube in fluid communication with the inflation tube lumen of the inflation tube.

The inflation tube generally has an outer surface and an inner surface. The inner surface of the inflation tube defines a lumen and an inflation lumen. The lumen and the inflation lumen may extend along at least a portion of the inflation tube. The inflation tube may further define a distal lumen opening in communication with the lumen. The inflation tube may further define an inflation lumen opening in communication with the inflation lumen. When present, the proximal tube may define a proximal lumen. The proximal lumen may extend between a proximal end and a distal end of the proximal tube. The distal end of the proximal tube may be secured to a proximal end of the inflation tube. When secured to the inflation tube, the proximal lumen of the proximal tube may be in fluid communication with the inflation lumen of the inflation tube. In one aspect, the distal end of the proximal tube may define a proximal tube notch. The proximal tube notch may receive a proximal end of the core wire extending from a proximal end of the inflation tube. The proximal end of the core wire may be secured within the proximal tube notch of the proximal tube. The core wire may define a longitudinal core wire mating surface and/or a perpendicular core wire mating surface. The longitudinal notch surface defining at least a portion of the notch may be secured to the longitudinal core wire mating surface. The perpendicular core wire mating surface of the core wire may be secured to a perpendicular notch surface of the proximal tube.

The torque tube is secured to the inflation tube. The torque tube may extend from the distal end of the inflation tube. The torque tube may be secured within the distal lumen opening of the inflation. The torque tube may define a torque tube lumen. In one aspect, the torque tube secured within the distal lumen opening of the lumen. The balloon secured over the distal portion of the inflation tube. The balloon defines an inflation chamber. The inflation chamber may be in fluid communication with distal inflation tube opening. The balloon may be slidably received over the torque tube at a distal end of the balloon. The balloon may include a sleeve. The sleeve may define a sleeve passage slidably receiving a portion of the torque tube. The sleeve may be integral with the balloon or may be a separate component secured to the balloon. In one aspect, the sleeve may be a disk peripherally secured to the balloon.

A core wire may extend through at least a portion of the core wire lumen. The core wire may extend from a distal end of the inflation tube. Typically, the core wire will extend from the distal core wire lumen opening and through the torque tube lumen.

Other features and advantages of the invention will become apparent from the following detailed description, and from the claims.

### BRIEF DESCRIPTION OF THE DRAWINGS

FIG. 1 illustrates a partial perspective view of an exemplary embodiment of a balloon apparatus in accordance with the present inventions;

FIG. 2 illustrates a detailed partial perspective view of an exemplary embodiment of a distal end of a balloon apparatus in accordance with the present inventions;

FIG. 3A illustrates a cross-section of a partial side view of an exemplary embodiment of a distal end of a balloon apparatus with an un-inflated balloon in accordance with the present inventions;

FIG. 3B illustrates a cross-section of a partial side view of an embodiment of a distal end of a balloon apparatus similar to the embodiment of FIG. 3A with a partially inflated balloon in accordance with the present inventions;

FIG. 3C illustrates a cross-section of a partial side view of an embodiment of a distal end of a balloon apparatus similar to the embodiment of FIG. 3A with a fully inflated balloon in accordance with the present inventions;

FIG. 4 illustrates a cross-section of a partial side view of another embodiment of a distal end of a balloon apparatus with an un-inflated balloon in accordance with the present inventions;

FIG. 5A illustrates a cross-sectional end view through an embodiment of an inflation tube in accordance with the present inventions at section 5-5 of FIGS. 3A to 4;

FIG. 5B illustrates a cross-sectional end view through an embodiment of an inflation tube in accordance with the present inventions at section 5-5 of FIGS. 3A to 4;

FIG. 6A illustrates a distal end view of an embodiment of a balloon apparatus in accordance with the present inventions;

FIG. 6B illustrates a distal end view of another embodiment of a balloon apparatus in accordance with the present inventions; and

FIG. 6C illustrates a distal end view of another embodiment of a balloon apparatus in accordance with the present inventions.

All Figures are illustrated for ease of explanation of the basic teachings of the present invention only; the extensions of the Figures with respect to number, position, relationship and dimensions of the parts to form the embodiment will be explained or will be within the skill of the art after the following description has been read and understood. Further, the exact dimensions and dimensional proportions to conform to specific force, weight, strength, flow and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they will typically be utilized by a physician or other user who is treating or examining a patient with an apparatus in accordance with the present invention.

### DETAILED DESCRIPTION OF THE INVENTION

The present inventions provide balloon apparatus 10 and associated methods for use in conjunction with medical catheters and medical guidewires. The figures generally illustrate embodiments of balloon apparatus 10 including aspects of the present inventions. The particular exemplary embodiments of the balloon apparatus 10 illustrated in the figures have been chosen for ease of explanation and understanding of various aspects of the present inventions. These illustrated embodiments are not meant to limit the scope of coverage but instead to assist in understanding the context of the language used in this specification and the appended claims. Accordingly, variations of balloon apparatus 10 for use with medical guidewires and medical catheters different from the illustrated embodiments may be encompassed by the appended claims.

Balloon apparatus 10 are generally configured to be introduced into a bodily lumen of a patient and have the balloon 18 positioned at a target location in the bodily lumen. The balloon 18 may then be inflated for a wide variety of medical purposes. Typically, the balloon 18 is inflated with an inflation media. The inflation media will frequently include various imaging compounds and may include various medicinal or other compounds that can be desirable in particular applications.

The balloon apparatus 10 in accordance with the present inventions includes at least an inflation tube 14, a torque member 16 and a balloon 18. The balloon 18 is secured to an end of the inflation tube 14 to permit the introduction of inflation media through the inflation tube 14 and into the balloon 18. The torque member 16 is secured to and extends from the distal end of the inflation tube 14. Typically, the balloon 18 is secured over the torque member 16 such that at least a portion of the torque member 16 is positioned within the inflation chamber 28 of the balloon 18. The balloon 18 slidably receives the torque member 16. The balloon 18 may further include a sleeve 38 to slidably receive the torque member 16. The sleeve 38 may form a seal to permit the sliding of the distal end of balloon 16 along the longitudinal axis of torque member 16 during inflation and/or deflation of balloon 18. The seal is typically configured to prevent medically significant amounts of inflation media from leaking from between the torque member 16 and the sleeve 38. In operation, the balloon 18 and the torque member 16 generally cooperate to reduce twisting and other deformation of the balloon 18 as the balloon 18 is positioned within a patient while permitting the balloon 18 to slide longitudinally along the torque member 16 primarily during inflation and deflation of the balloon 18.

In addition, the balloon apparatus 10 may include a core wire 20. The core wire 20 can positioned through the inflation tube 14 and torque member 16. In one aspect, the core wire 20 extends distally from the distal end of torque member 16. The core wire 20 may confer desired performance characteristics such as a desired torquability or a desired pushability to the balloon apparatus 10.

As illustrated throughout the figures for exemplary purposes, balloon apparatus 10 include an inflation tube 14, a torque member 16 and a balloon 18. Balloon apparatus 10 in accordance with the present inventions may further include a proximal tube 12 and/or a core wire 20. The inner surface 74 of the inflation tube 14 may define an inflation lumen 24 and a lumen 30. The balloon 18 can be secured over an outer surface 64 of the inflation tube 14 at the distal end 214 of the inflation tube 14. An inflation chamber 28 is typically defined, at least in part, by an inner surface 78 of the balloon 18. The inflation chamber 28 can be in fluid communication with the inflation lumen 24. Typically, a distal inflation opening 44 defined in distal end 214 of the inflation tube 14 communicates fluid from the inflation lumen 24 into the inflation chamber 28 of the balloon 18. In various aspects, the distal inflation tube opening 44 may be located at or near the distal end 214 of the inflation tube 14. A proximal inflation tube opening 34 in fluid communication with the inflation lumen 24 may be located at or near the proximal end 114 of the inflation tube 14 for the introduction of inflation media from a location remote from the balloon 18. The torque member 16 is secured to and extends from the distal end 214 of the inflation tube 14. Typically, a proximal end 116 of the torque member 16 is secured within a distal lumen opening 50 of a lumen 30. The torque member 16 may generally extend from the inflation tube 14 in an orientation parallel or coaxial with the longitudinal axis of the inflation tube 50. The torque member 16 typically extends into the inflation chamber 28 of the balloon 18. The distal end 218 of balloon 18 is slidably secured over at least a portion of the torque member 16. In one aspect, the distal end 218 of the balloon slides along the torque member 16 during inflation and deflation of the balloon 18. A stop 36 may be provided at the distal end of the torque member 16 to prevent the distal end of the balloon 18 from extending beyond the end of the torque member 16. In various aspects, the balloon 18 may further include a sleeve 38 to slidably receive the torque member 16. The sleeve 38 may be integral with the balloon 18 or a separate structure secured to the balloon 18. When present, the core wire 20 may be received within the lumen 30. The core wire 20 may further extend from the lumen 30 through distal lumen opening 50 and into a torque tube lumen 30. In one aspect, at least a portion of the core wire 20 may extend from the distal end 216 of the torque member 16. The core wire 20 may be provided to confer the desired torquability and pushability to the region of the inflation tube 14 through which it extends. In one aspect, the core wire may be secured to the inflation tube 14 at one or more locations along its length. In other aspects, the core wire 20 may be secured in lumen 30 or within other structures independent from lumen 30. When present, the proximal tube 12 is secured to the proximal end of the inflation tube 14. A proximal lumen 22 of the proximal tube 12 is typically in fluid communication with the inflation lumen 24 of the inflation tube 14. To secure the inflation tube 14 to the proximal tube 12, the inflation tube 14 may define a distal notch 62 to receive a proximal notch 52 of the proximal tube 12. For purposes of the present inventions, the term “secured to” means that the distal tubular portion is attached to main proximal portion by a suitable method such as, for example, by welding, brazing, heat shrinking, or gluing among other methods. For purposes of description, balloon apparatus 10 generally should be considered to have longitudinal axis 300 defined along its length as generally illustrated in the figures regardless of any curvature in the balloon apparatus 10.

In particular, a proximal tube 12 is configured to guide and position portions of the inflation tube 14 within a patient. The proximal tube 12 may function as the point of articulation for a user as the inflation tube 14 is introduced into a bodily lumen of a patient. The proximal lumen 22 of the proximal tube 12 extends over at least a portion of the length of the proximal tube 12. The proximal lumen 22 of the proximal tube 12 may extend longitudinally within the proximal tube 12 from a first proximal tube opening 32 to a second proximal tube opening 42 defined by the proximal tube 12. The proximal lumen 22 is generally configured to receive a fluid, such as, for example, inflation media, and communicate the fluid at least to the second proximal tube opening 32. The proximal tube lumen 22 is typically in fluid communication with the inflation lumen 24 of the inflation tube 14.

The proximal tube 12 may further define a proximal notch 52 at a distal end 212 of the proximal tube 12 which is configured to be secured to a distal notch 52 of the inflation tube 14. The proximal notch 52 generally extends from the distal end 212 of the proximal tube 12 to a location along the proximal tube 12 which is proximal to the distal end 212 of the proximal tube 12. The proximal notch 52 is generally shaped to receive the distal notch 62 at a first end 214 of inflation tube 14. The proximal notch 52 may extend into the proximal lumen 22. When the proximal tube 12 is secured to the inflation tube 14, the proximal lumen 22 and the inflation lumen 24 together may form a continuous passage extending between a proximal end 112 of proximal tube 12 and a distal inflation tube opening 44 of inflation tube 14.

The proximal tube 12 may be made from a variety of materials including polymers, metals, and various composite materials. In one aspect, the proximal tube 12 is made of a stainless steel. In another aspect, the proximal tube 12 is made of nitinol. Typically, the proximal tube 12 is configured to have a desired elastic range. The proximal tube 12 may be configured to have a desired balance of longitudinal stiffness and torsional rigidity based on the characteristics of the inflation tube 14. The longitudinal stiffness, at least in part, dictates the push characteristics for the proximal tube 12. The torsional rigidity, at least in part, dictates the precision of the rotational control provided by the proximal tube 12.

The proximal tube 12 may have various outside diameters 312 and lengths depending on the particular application for the balloon apparatus 10. Generally, the proximal tube 12 is configured to at least support inflation of the associated balloon 18. The particular configuration of proximal tube 12 may also depend upon whether or not the proximal tube 12 is intended primarily for use as a balloon catheter or as a wire support for other guidewires or catheters. For use primarily as a balloon catheter, a balloon apparatus 10 may be configured to support larger volumes of fluid than when the balloon apparatus 10 used as a delivery rail for other devices. In such applications for balloon inflation, the proximal tube 12 may have an outside diameter 312 of about 0.024 inches and a lumen diameter of about 0.019 inches. This outside diameter 312 can provide the desired torsional rigidity without being too longitudinally stiff. The inside diameter 412 of the proximal lumen 22 may be selected to provide a desire inflation/deflation time. For intercranial applications where the insertion point is in the femoral artery, the length of the proximal tube 12 can be about 110 centimeters. A proximal tube 12 of this length may keep the proximal tube 12 in the straight portion of the guide. For use of balloon apparatus 10 as a guide wire, the outside diameter 312 can be around 0.014 inches. For other applications requiring access to smaller bodily lumen, an outside diameter 312 of less than 0.014 inches may be used. In one exemplary embodiment, the proximal tube 12 of the balloon apparatus 10 can have an outside diameter 312 of the order of 0.014 inches and a wall thickness of the order of 0.002 inches to maximize the inside diameter 412 of the proximal lumen 22. The proximal tube 12 can be between about 165 cm to about 205 cm in length and although flexible, have a stiffness of about 50-100 N-mm² to impart sufficient lateral stiffness and torque transmission capabilities along its length.

In particular, the inflation tube 14 is configured to facilitate the positioning of the distal end 114 of inflation tube 14 at a desired location within a bodily lumen of a patient and to permit the inflation of the balloon 18. The inflation tube 14 may be used without an associated proximal tube 12 or may be secured to the distal end 214 of a proximal tube 12. When balloon apparatus 10 includes a proximal tube 12, the proximal end 114 of the inflation tube 14 is typically secured to a distal end 212 of the proximal tube 12. In one aspect, the proximal tube 12 may be secured to the inflation tube 14 using apparatus and methods as disclosed in U.S. patent application Ser. No. 11/333,045 entitled Medical Catheters and Methods the disclosure of which is hereby incorporated by reference in its entirety. The inflation tube 14 defines at least one inflation lumen 24 to permit the communication of fluids along at least a portion of the length of the inflation tube 14. An inner surface 74 of inflation tube 14 defines an inflation lumen 24 extending along at least a portion of the length of inflation tube 14. The inflation tube 14 includes a balloon 18 secured at or near the distal end 214 of the inflation tube 14 in fluid communication with the inflation lumen 24. An inner surface 74 of the inflation tube 14 may also define a lumen 30 which extends along at least a portion of the length of the inflation tube 14. The inflation tube 14 may further include a core wire 20 extending within the inflation tube 14 over at least a portion of its length. In one aspect, the core wire 20 is positioned within the lumen 30 of the inflation tube 14.

The inflation lumen 24 of the inflation tube 14 typically extends over at least a portion of the length of the inflation tube 14. The inflation lumen 24 is generally configured to communicate a fluid along a portion of the length of the inflation tube 14. In one aspect, the inflation lumen 24 may be configured to communicate a fluid from a proximal end 114 of inflation tube 14 to a distal end 114 of the inflation tube 14 or to a location adjacent to the distal end 214 of inflation tube 14. The inflation lumen 24 of inflation tube 14 may extend longitudinally within the inflation tube 14 from a first inflation tube opening 34 to a distal inflation tube opening 44 defined by the inflation tube 14. The proximal inflation tube opening 34 is typically in communication with the inflation lumen 24 at or near the proximal end 114 of the inflation tube 14. The distal inflation tube opening 44 is typically in communication with the inflation lumen 24 at or near the distal end 214 of the inflation tube 14. The inflation lumen 24 may receive a fluid, such as for example inflation media, through a proximal inflation tube opening 34 and communicate the fluid to at least the distal inflation tube opening 44. When balloon apparatus 10 includes a proximal tube 12, the inflation lumen 24 is typically in fluid communication with the proximal lumen 22 of the proximal tube 12.

The inflation tube 14 may be made from a range of materials and configurations depending upon the intended use for the resultant balloon apparatus 10. In one aspect, the tube may be a metal, such as, for example, stainless steel or nitinol. In another aspect, the inflation tube 14 can be made from one or more polymers such as polyethylene, nylon, polyimide, among others. The materials are generally selected to provide a desired balance of longitudinal stiffness and torsional rigidity based on the characteristics of the inflation tube 14 and, when a core wire 20 is provided, in combination with a core wire 20 extending along at least a portion of the length of the inflation tube 14.

The inflation tube 14 typically has an outside diameter 314 which is the same or smaller than the outside diameter 312 of the proximal tube 12. For use primarily as a balloon catheter, the inflation tube 14 may have an outside diameter 314 of 0.024 inches. The inflation lumen 24 may be configured with as large a cross-sectional area as large as possible given the size and particular application for the balloon apparatus 10. In one exemplary embodiment, the inflation tube 14 of a balloon apparatus 10 has a length from about 15 cm to about 25 cm. The inflation tube 14 has an outside diameter 314 of about 0.014 inches and is secured to a proximal tube 12 having the same outside diameter 312. Inflation tube 14 may have a stiffness of about 25-50 N-mm² or less, to impart the desired flexibility to balloon apparatus 10. Additionally, the flexibility of balloon apparatus 10 may be varied by progressively annealing either a portion, for example, only inflation tube 14, or the entire length of balloon apparatus 10.

In particular, the torque member 16 is typically an elongated member extending distally from the distal end 214 of the inflation tube 14. The torque member 16 is secured to the distal end of the inflation tube 14 or in some aspects may be integral with the inflation tube 14. Typically, a proximal end 116 of the torque member 16 is secured within a distal lumen opening 50 of a lumen 30. The torque member 16 may generally extend from the inflation tube 14 in an orientation parallel or coaxial with the longitudinal axis of the inflation tube 50. The torque member 16 is configured to permit the longitudinal movement of a balloon 18 along its longitudinal axis during inflation and/or deflation. Accordingly, the outer surface 76 may be configured to both sealably and slidably engage an aspect of balloon 18.

The torque member 16 may be further configured to inhibit the deformation of a balloon 18 while positioning the balloon apparatus 10 in a patient. In one aspect, the torque member 16 may inhibit the deformation of the balloon 18 by preventing the rotation of the distal end 218 about the longitudinal axis of the inflation tube 14. When the longitudinal axis of the torque member 16 is coaxial with the longitudinal axis of the inflation tube 14, the torque tube 16 may prevent the deformation of balloons 18 by having the exterior surface 66 of torque member 16 define a non-circular transverse cross sectional shape which rotatably engages the distal end 218 of the balloon 18 to prevent rotation while permitting the longitudinal sliding of the balloon 18 over the torque member 16. When the longitudinal axis of torque member 16 is parallel and not coaxial to the longitudinal axis of the inflation tube 14, the torque tube 16 may prevent the deformation of balloons 18 by providing a moment arm between the longitudinal axis of the torque member 16 and the longitudinal axis of the balloon 18/inflation tube 14 which inhibits rotation while permitting the longitudinal sliding of the balloon 18 over the torque member 16. The moment arm being related to the distance between the two longitudinal axes.

The torque member 16 extends through at least a portion of the inflation chamber 28 of balloon 18. A distal end 218 of balloon 18 is slidably secured over at least a portion of the torque member 16 such that the distal end 218 of balloon 18 may slide along torque member 16 during inflation or deflation of the balloon 18. A stop 36 may be provided at the distal end of the torque member 16 to prevent the distal end of the balloon 18 from extending beyond the end of the torque member 16. The stop 36 may be in the form of a protuberance extending from the outer surface 76 of the torque member 16 or may be a circumferential ring secured about or integral with the torque member 16. The outer surface 76 of torque member 16 may have a transverse cross-sectional shape of a circle, or may be otherwise shaped such as in the shape of an oval or having a longitudinal rib as will be recognized by those skilled in the art to prevent the rotation of the balloon 18 about the torque member 16.

In certain aspects, an inner surface 76 of the torque member 16 may define a torque member lumen 26. In certain aspects, the torque member lumen 26 may receive a portion of a core wire 20. An adhesive 80 may secure the core wire 20 within the torque member lumen 26.

The torque member 16 may be made from a range of materials and configurations depending upon the intended use for the resultant balloon apparatus 10. In one aspect, the torque member 16 may be a metal, such as, for example, stainless steel or nitinol. In another aspect, the torque member 16 can be made from one or more polymers such as polyethylene, nylon, polyimide, among others. The materials are generally selected to provide a desired balance of longitudinal stiffness and torsional rigidity based on the characteristics of the torque member 16 and, when present, in combination with a core wire 20.

The torque member 16 typically has an outside diameter 316 which is the same or smaller than the outside diameter 316 of the inflation tube 16. For use primarily as a balloon catheter, the torque member 16 may have an outside diameter 316 of 0.0?? inches. In one exemplary embodiment, the inflation tube 14 of a balloon apparatus 10 has a length from about 15 cm to about 25 cm. The inflation tube 14 has an outside diameter 314 of about 0.014 inches and is secured to a proximal tube 12 having the same outside diameter 312. The torque member 16 may have a stiffness of about ??-?? N-mm² or less, to impart the desired flexibility to balloon 18.

A balloon 18 may be provided at or near the distal end 214 of the inflation tube 14 for inflation within the bodily lumen of a patient. In one aspect, a proximal end 118 of a balloon 18 may be positioned at or near the distal end 214 of the inflation tube 14. The balloon 18 defines an inflation chamber 28 to receive inflation media from the inflation lumen 24 of the inflation tube 14. In one aspect, the inflation chamber 28 is in fluid communication with the inflation lumen 24. In one aspect, the balloon 18 may be positioned over at least one distal inflation tube opening 44 which is in fluid communication with the inflation lumen 24.

The balloon 18 includes a sleeve 38 that is slidably received over the torque member 16. The sleeve 38 forms a seal to permit the inflation of balloon 18 and is typically configured to prevent medically significant amounts of inflation media from leaking from between the torque member 16 and the sleeve 38. The sleeve 38 defines a sleeve passage 58 to receive a portion of the torque member 16. The torque member 16 extends from the inflation tube 14 into the inflation chamber 28 of the balloon 18 and through the sleeve passage 58 of the sleeve 38. The sleeve passage 58 typically has a shape which corresponds to the cross-sectional shape of the torque member 16 at the region of the core wire 20 passing through the sleeve 38. As the balloon 18 inflates, sleeve 38 typically slides distally along a portion of torque member 16 or associated structure. For deflation, the inflation lumen 24 receives fluid from the balloon 18. As the balloon 18 deflates, the sleeve 38 typically slides proximally along a portion of torque member 16 or associated structure.

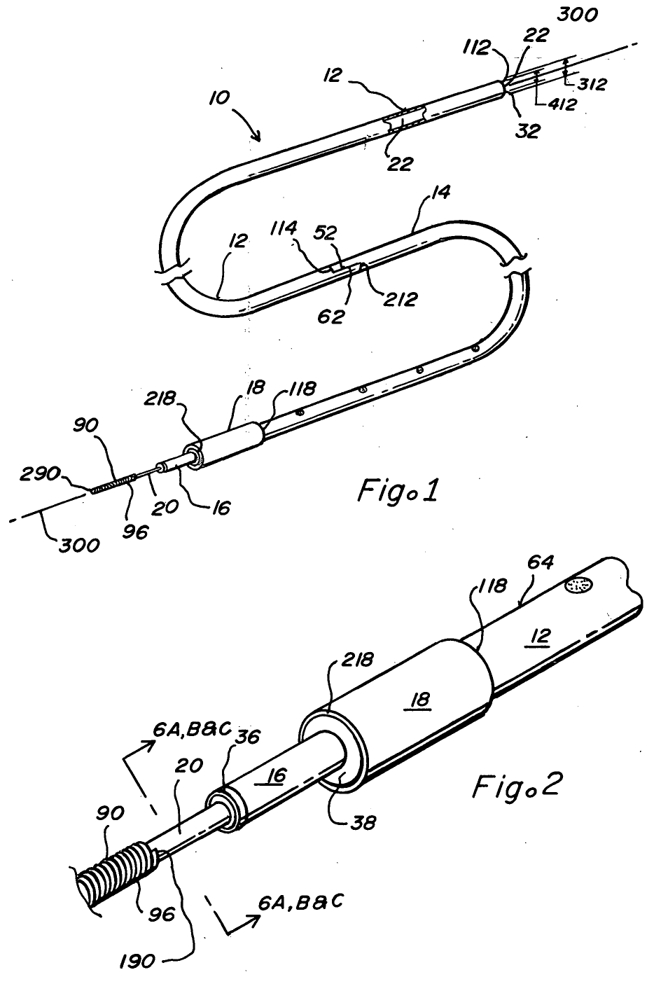

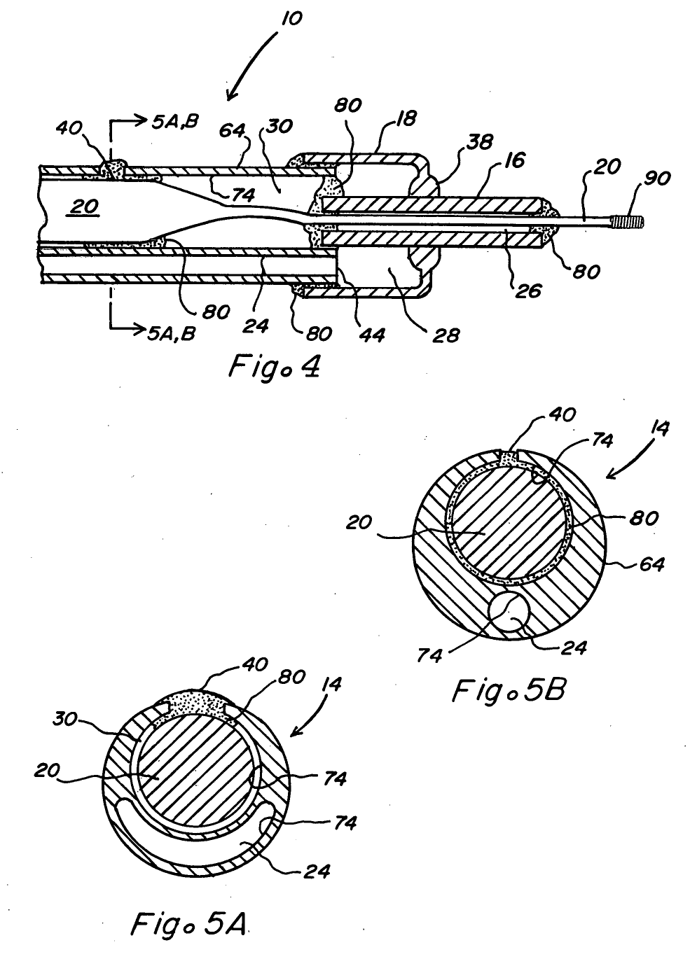

The sleeve 38 may be a separate structure secured to the balloon 18, as illustrated in FIGS. 1 to 3C, for exemplary purposes, or integral with the balloon 18, as illustrated in FIG. 4 for exemplary purposes. When integral, the sleeve 38 may be a thickened or reinforced region of the balloon 18 that resists deformation and leaking upon introduction of inflation media into the expansion chamber 28 and inflation of the balloon 18. When a separate structure, the sleeve 38 can be a disk 48 which defines the sleeve passage 58. The disk 48 may be peripherally secured to the balloon 18. The disk 48 may be generally expandable and elastic, it may be generally rigid, or it may be otherwise configured. However, the sleeve passage defined by the disk 48 is configured to resist deformation and leaking upon introduction of inflation media into the expansion chamber 28 and inflation of the balloon 18. A lubricious coating 82 may be provided between the sleeve 38 and the core wire 20 to reduce frictional forces between the sleeve 38 and core wire 20 during inflation and deflation as the sleeve 38 slides along the core wire 20. In one aspect, the lubricious coating 82 is provided over at least a portion of the sleeve passage 48. In another aspect, the lubricious coating 82 is provided over at least a portion of the length of the core wire 20.

Depending upon the application for the balloon apparatus 10, the balloon 18 may be configured with a wide range of physical specifications and performance characteristics as will be recognized by those skilled in the art upon review of the present disclosure. In one aspect, the balloon 18 may be either compliant or non-compliant. For various applications, the balloon 18 may be configured and sized to provide the desired inflated diameter and length for a treatment and location. In neurovascular applications, the target vessel diameters may range from as large as 10 to 12 millimeters to as small as 2 to 3 millimeters. The balloon 18 may be configured to circumferentially contact the walls of these vessels and may be provided in a variety of different lengths depending on the treatment and/or purpose of the balloon. In compliant embodiments, the balloon 18 may be made from silicone. For neurovascular applications, silicone may provide additional therapeutic benefits relating to spasms that will be recognized by those skilled in the art upon review of the present disclosure. When silicone is used, the silicone material may have a durometer of about 20 to 30. For neurovascular applications, this may give the balloon apparatus 10 the correct ‘feel’ when the balloon is inflated to a pressure of about 1 atmosphere.

When present, the core wire 20 may be secured within the inflation tube 14 and typically extends over at least a portion of the length of the inflation tube 14. The core wire 20 may confer a desired balance of longitudinal stiffness and torsional rigidity characteristics to the inflation tube 14 through which the core wire 20 extends. Further, the core wire 20 may be secured to or extend into the proximal tube 12 when a proximal tube 12 is included in the balloon apparatus 10. When secured to the proximal tube 12 the core wire 20 may transmit the torquing and pushing of the proximal tube 12 by a user to at least the distal portions of the inflation tube 14. In other aspects, the core wire 20 may be used to, at least in part, secure the inflation tube 14 to the proximal tube 12 of a balloon apparatus 10. When secured along a length of the proximal tube 12, the core wire 20 may confer a desired balance of longitudinal stiffness and torsional rigidity characteristics to the portion of the proximal tube 12 through which the core wire 20 extends.

The core wire 20 may be secured within the lumen 30 of the distal tube 14. In one aspect, the core wire 20 may be secured at one or more discrete locations along the length of the core wire 20. The core wire 20 may be secured one or more discrete locations by introducing an adhesive through one or more transverse passages 40 extending between the outer surface 64 and a portion of inner surface 74 defining lumen 30. In other aspects, the core wire 20 may be rotatably and/or slidably received within the inflation lumen 24 of the inflation tube 14.

The core wire 20 is typically a metal wire having a circular transverse cross-section as shown in FIG. 8A for exemplary purposes. The core wire 20 is typically made of a rigid but elastic material. Although the core wire 20 is typically made from stainless steel or nitinol, the core wire 20 may be formed from other metals, polymers or composite materials as will be recognized by those skilled in the art upon review of the present disclosure. The core wire 20 is typically a solid wire, however the core wire 20 may be hollow along at least a portion of its length. The core wire 20 may also be in the form of a wound cable, a braided filament, or otherwise alternatively configured as will be recognized by those skilled in the art upon review of the present disclosure. In other aspects, the core wire 20 may be tapered along the distal portion of the core wire such that the decreasing diameter provides greater flexibility to the region of the core wire 20 extending beyond the distal end 214 of the inflation tube 14.

For intercranial applications, the core wire 20 may be about 40 centimeters long when the insertion point is the femoral artery. In an exemplary embodiment where the proximal tube 12 has an outside diameter 312 of 0.014 inches, the proximal end 116 of the core wire 20 can have a diameter 316 of about 0.009 inches where it attaches to the proximal tube 12. The core wire 20 may include several reductions in outside diameter 316 toward the distal end 216 of core wire 20. In this aspect, the core wire 20 may have a diameter of about 0.004 inches at the distal end 216 of the core wire 20.

An atraumatic tip 90 may be attached to the distal end 114 of the torque member 16 or the core wire 20. The atraumatic tip 90 generally provides a soft, gentle bumper for the distal end 216 of the torque member 16 or the core wire 20. The atraumatic tip 90 may include a coil 96. The coil 96 may be about 2 cm long and about 0.014 inches in diameter. The coil 96 can be made of 0.002 inches in diameter radio opaque material, preferably platinum. However, other materials known in the art can be used as well. A shaping ribbon may be positioned within the coil 96. The shaping ribbon is typically constructed from a metal and can serve several important functions. The shaping ribbon may serve as a bendable beam to more easily permit a user to induce a curved shape in the atraumatic tip 90 to direct the balloon apparatus 10 through a bodily lumen of a patient. Further, the shaping ribbon may improve the safety of a balloon apparatus 10 by not allowing the coils 96 of the atraumatic tip 90 to stretch out if a portion of the atraumatic tip 90 becomes lodged or otherwise hung up in the bodily lumen of a patient. The proximal end of the shaping ribbon may be attached to the distal end 216 of the core wire 20 and/or the proximal ends of the coils 96. The distal end of the shaping ribbon may be secured to the distal end of the coils 96. The thickness of the shaping ribbon for intercranial applications is typically about 0.002 inches by 0.004 inches. The shaping ribbon is made from a material having the desired combination of ductility and elasticity. Stainless steel of a proper temper is commonly used to provide these characteristics. The coil 96 may terminate in a rounded cap as to be generally atraumatic to the wall of a bodily lumen.

As illustrated for exemplary purposes, FIGS. 1 and 2 illustrate an embodiment of a balloon apparatus 10 in accordance with the present inventions including both a proximal tube 12 and an inflation tube 14. FIG. 1 illustrates a general view of portions of the entire length of a balloon apparatus 10. FIG. 2 illustrate a more detailed view of a distal portion of a similar balloon apparatus 10. The proximal tube 12, inflation tube 14 and torque member 16 are illustrated as having a circular cross-section for exemplary purposes. The illustrated embodiment includes a passage extending from a proximal end 112 of the proximal tube 12 to a distal inflation tube opening 44 underlying the balloon 18 at a region proximal to the distal end 214 of the inflation tube 14 to communicate inflation media from the proximal end 112 of the proximal tube 12 to the inflation chamber 28 of the balloon 18. The passage is formed by connecting the proximal tube 12 to the inflation tube 14 such that the proximal lumen 22 of the proximal tube 12 is in fluid communication with the inflation lumen 24 of the inflation tube 14. As illustrated in FIG. 1, the proximal tube 12 is secured to the inflation tube 14 by overlapping a proximal notch 52 in the proximal tube 12 with a distal notch 62 in the inflation tube 14.

A portion of the torque tube 16 is shown extending through a sleeve passage 58 of sleeve 38. The sleeve 38 is illustrated for exemplary purposes as a disk 48 defining an axially positioned sleeve passage 58. A core wire 20 is shown extending distally from a torque tube lumen 26 of torque tube 16. An atraumatic tip 90 is shown secured to the distal end 216 of core wire 20. In the illustrated embodiment, the inflation tube 14 is generally configured to be directed through a bodily lumen within a patient by a physician manipulating the proximal tube 12 and, once properly positioned, to have the balloon 18 inflated for diagnostic or therapeutic purposes.

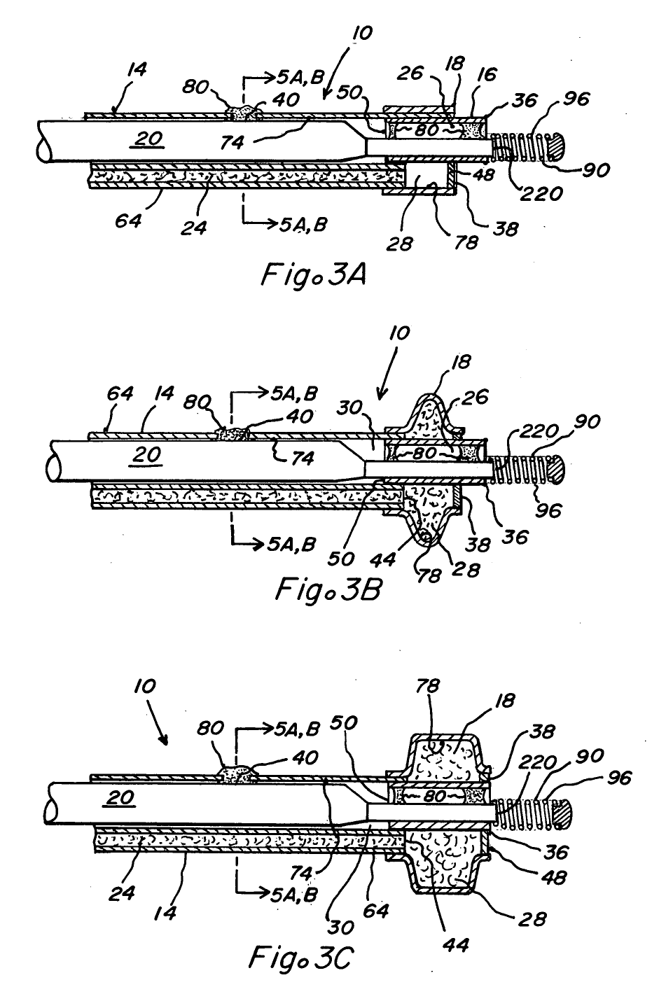

FIGS. 3A to 3C illustrate a cross-section of the distal portion of an exemplary embodiment in accordance with the present invention. FIGS. 3A to 3C show an exemplary cross-section at the distal end 214 of the inflation tube 14 including a torque member 16 and a balloon 18. The balloon is shown at increasingly expanded configurations from a substantially un-inflated configuration in FIG. 5A to a substantially fully-inflated configuration in FIG. 5C.

FIG. 3A illustrates an exemplary balloon 18 having a proximal end 118 secured over the distal end 214 of the inflation tube 14 with the balloon 18 in a substantially un-inflated configuration. The proximal end 118 of the balloon 18 is in fluid communication with the lumen 24 through a plurality of inflation tube openings 44 positioned proximal to the distal end 214 of the inflation tube 14. As illustrated for exemplary purposes, an adhesive 80 is used to secure the balloon 18 to the inflation tube 14. Welding, shrinking, expanding, mechanical bands, or other methods or devices may alternatively be used to secure the balloon 18 to the inflation tube 14. A region of the core wire 20 proximal to that extending from the distal end 214 of inflation tube 14 is secured within inflation tube 14 with an adhesive 81 introduced through a transverse passage 40. The adhesive 80 is shown extending about the distal portion of the core wire 20 to secure the core wire 20 to the inflation tube 14. The guide tube 16 is positioned within the lumen opening 50 of lumen 30 and may also be secured in position with an adhesive 80 or other materials or methods as will be recognized by those skilled in the art upon review of the present disclosure. The distal portion of core wire 20 extends through a guide tube lumen 26. The core wire 20 is secured within the guide tube lumen 26 with an adhesive 80. In another aspect, a sealant 80 may be provided to seal the guide tube lumen 26. An inflation tube opening 44 communicates with lumen 34 at a distal end 214 of the inflation tube 14. As illustrated, the distal inflation tube openings 44 communicate inflation media into a proximal portion of the inflation chamber 28. A portion of guide tube 16 extends into and through an inflation chamber 28 defined by the balloon 18 and passes through a sleeve passage 58 of sleeve 38. The sleeve 38 is shown as a disk 48 peripherally secured to the balloon 18 to form the inflation chamber 28. As illustrated, the sleeve 38 is generally configured to allow the distal end 218 of balloon 18 to slide proximally and distally along the guide tube 16 as the balloon 18 is inflated and deflated, respectively. A lubricious coating 82 is provided on the sleeve 38 within the sleeve passage 58 for exemplary purposes.

FIG. 3B illustrates the balloon of FIG. 3A in a partially inflated configuration. The inflation media introduced in the inflation chamber 28 through the inflation lumen 24 and the inflation tube opening 44 is shown first inflating the proximal end 118 of the balloon 18. As the balloon 18 is inflated, the distal end 218 of the balloon is displaced distally along guide tube 16 as the sleeve 38 slides along the guide tube 16.

FIG. 3C illustrates the balloon 18 of FIGS. 3A and 3B in a substantially fully inflated configuration. The inflation media introduced in the inflation chamber 28 through the inflation lumen 24 and the plurality of distal inflation tube opening 44 is shown having inflated the balloon 18 from the proximal end 118 to the distal end 218 of the balloon 18. With the balloon fully inflated, the distal end 218 of the balloon has been displaced distally along the guide tube 16 to about the location of stop 36 at the distal end 216 of the guide tube 16 for exemplary purposes. As inflation media is removed from the inflation chamber 28, the distal end 218 of the balloon 18 may move proximally along the guide tube 16 until the balloon 18 is in a relaxed and deflated condition.

FIG. 4 illustrates a cross-section of an exemplary embodiment of apparatus in accordance with the present invention. FIG. 4 shows an exemplary cross-section the distal end 214 of the inflation tube 14 including a guide tube 16 and a balloon 18 in a substantially un-inflated configuration. The inflation tube 14 is again illustrated as having a circular cross-section for exemplary purposes. The illustrated embodiment may include a passage in the form of inflation lumen 24 extending from a first end 114 of the inflation tube 14 to a distal inflation tube opening 44 underlying the balloon 18 at a region proximal to the second end 214 of the inflation tube 14 to communicate inflation media from the proximal end 112 of the proximal tube 12 to the inflation chamber 28 of the balloon 18. A portion of a guide tube 16 is shown extending through a sleeve passage 58 of sleeve 38 secured to balloon 18. A core wire 20 extends through a guide member lumen 26 of guide member 16. An atraumatic tip 90 is shown secured to the distal end 216 of core wire 20.

The balloon 18 illustrated in FIG. 4 has a proximal end 118 secured over the distal end 214 of the inflation tube 14. The proximal end 218 of the balloon 18 is in fluid communication with the lumen 24 through an inflation tube opening 44 at the distal end 214 of the inflation tube 14. As illustrated for exemplary purposes, an adhesive 80 is used to secure the balloon 18 to the inflation tube 14. Welding, shrinking, expanding, mechanical bands, or other methods or devices may alternatively be used to secure the balloon 18 to the inflation tube 14. A guide tube 16 is secured in a lumen opening 50 of lumen 30. The guide tube extends into the inflation chamber 28 defined by the balloon 18 and passes through a sleeve passage 58 of sleeve 38. A core wire 20 is also secured within lumen 30. A portion of the core wire 20, shown tapered to a reduced diameter for exemplary purposes, extends into and through the guide tube lumen 26.

As illustrated in FIG. 4, an atraumatic tip 90 is secured to the distal end 216 of core wire 20. The atraumatic tip includes a coil 96. As illustrated, the sleeve 38 is generally configured to allow the distal end 318 of balloon 18 to slide proximally and distally as the balloon 18 is inflated and deflated respectively. A lubricious coating 82 may be provided on the outer surface of the torque tube 16.

FIGS. 5A and 5B illustrate embodiments of a cross-section through the inflation tube 14 at section lines 5-5 of FIGS. 3A to 4. As illustrated in FIG. 5A, the transverse passage 40 may 40 has received an adhesive 80 around a portion of the periphery of core wire 20 to secure a core wire 20 within the lumen 30. The inflation lumen 24 has been configured in a rounded crescent shape to more fully optimize the fluid flow through the inflation lumen 24. As illustrated in FIG. 5B, the transverse passage 40 may 40 has received an adhesive 80 around the periphery of core wire 20 to secure a core wire 20 within the lumen 30. The inflation lumen 24 has been configured in a shape to allow the passage of fluid through the inflation lumen 24.

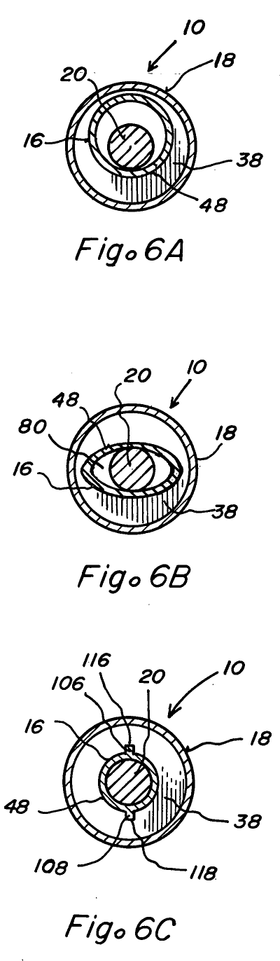

FIGS. 6A, 6B and 6C illustrate exemplary distal end views of balloon apparatus 10 in accordance with the present inventions. As illustrated in FIG. 6A, the guide tube 16 has a longitudinal axis parallel but not coaxial to the longitudinal axis of the inflation tube 14. Accordingly, balloon 18 is inhibited from rotating about the longitudinal axis during positioning of the balloon apparatus 10 within a patient. As illustrated in FIG. 6B, the guide tube 16 has a longitudinal axis coaxial to the longitudinal axis of the inflation tube 14. The guide member 16 however has a non-circular cross-sectional shape configured as an oval and received through a substantially oval shaped sleeve passage 48. Accordingly, balloon 18 is inhibited from rotating about the longitudinal axis during positioning of the balloon apparatus 10 within a patient. As illustrated in FIG. 6C, the guide tube 16 has a longitudinal axis coaxial to the longitudinal axis of the inflation tube 14. The guide member 16 however has a circular cross-sectional shape including a first longitudinal rib 106 and a second longitudinal rib 108 received a first groove 116 and a second groove 118 in a circular shaped sleeve passage 48. Accordingly, balloon 18 is inhibited from rotating about the longitudinal axis during positioning of the balloon apparatus 10 within a patient.

To use a balloon apparatus 10 in accordance with the present invention, a user may insert the distal end of balloon apparatus 10 into a bodily lumen of a patient using, for example, the Seldinger technique. The balloon apparatus 10 is guided through the bodily lumen to a location within the patient requiring treatment. As balloon apparatus 10 is guided through the patient, a user can manipulate the proximal tube 12 or the proximal end 114 of the inflation tube 14 to direct the distal end 214 of the inflation tube 14 through the bodily lumen. When the distal end 214 of the inflation tube 14 is positioned at or near the location within the bodily lumen requiring treatment, the user may initiate the desired treatment. In embodiments where the balloon apparatus 10 includes a balloon 18 at or near the distal end 214 of the inflation tube 14, the balloon 18 may be inflated to a desired size and/or pressure to affect the desired treatment. An balloon apparatus 10 including a balloon 18, properly sized and configured, may enable a user to access more distal or tortuous regions of the body. For example, when the distal portion of the balloon apparatus 10 has an outside diameter of around 0.014 inches, small lumen such as various arteries and veins in the brain and heart may be more easily accessed for diagnosis and/or treatment of the particular lumen or region.

Balloon apparatus 10 may further be used to guide surgical, therapeutic or diagnostic instruments over balloon apparatus 10 to access a desired location in a bodily lumen. When the instrument is positioned at the desired location within the bodily lumen, at least one surgical, therapeutic or diagnostic procedure using the instrument is performed. The instrument may be removed and replaced with a different instrument as required by the treatment, diagnosis, or surgical procedure being performed by the user.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

What is claimed is:

1. A torquable balloon apparatus for accessing a bodily lumen of a patient, comprising:
an inflation tube having an outer surface and an inner surface, the inner surface defining a lumen and an inflation lumen, the lumen and the inflation lumen extending along at least a portion of the inflation tube, the inflation tube defining a distal lumen opening in communication with the lumen, the inflation tube defining an inflation lumen opening in communication with the inflation lumen;
a torque tube defining a torque tube lumen, the torque tube secured to the inflation tube and extending from the distal end of the inflation tube; and
a balloon defining an inflation chamber, the balloon secured over the distal portion of the inflation tube with the inflation chamber in fluid communication with distal inflation tube opening, the balloon slidably received over the torque tube at a distal end of the balloon.

2. A balloon apparatus, as in claim 1, further comprising the balloon including a sleeve, the sleeve defining a sleeve passage slidably receiving a portion of the torque tube.

3. A balloon apparatus, as in claim 2, further comprising the sleeve integral with the balloon.

4. A balloon apparatus, as in claim 3, further comprising a proximal tube defining a proximal lumen extending between a proximal end and a distal end of the proximal tube, the distal end of the proximal tube secured to a proximal end of the inflation tube with the proximal lumen of the proximal tube in fluid communication with the inflation lumen of the inflation tube.

5. A balloon apparatus, as in claim 2, further comprising the sleeve secured to the balloon.

6. A balloon apparatus, as in claim 5, the sleeve comprising a disk peripherally secured to the balloon.

7. A balloon apparatus, as in claim 6, further comprising a proximal tube defining a proximal lumen extending between a proximal end and a distal end of the proximal tube, the distal end of the proximal tube secured to a proximal end of the inflation tube with the proximal lumen of the proximal tube in fluid communication with the inflation lumen of the inflation tube.

8. A torquable balloon apparatus as in claim 1, further comprising a core wire extending through at least a portion of the core wire lumen and extending from a distal end of the inflation tube from the distal core wire lumen opening, the core wire extending through the torque tube lumen.

9. A balloon apparatus, as in claim 8, further comprising the balloon including a sleeve, the sleeve defining a sleeve passage slidably receiving a portion of the torque tube.

10. A balloon apparatus, as in claim 9, further comprising the sleeve integral with the balloon.

11. A balloon apparatus, as in claim 10, further comprising a proximal tube defining a proximal lumen extending between a proximal end and a distal end of the proximal tube, the distal end of the proximal tube secured to a proximal end of the inflation tube with the proximal lumen of the proximal tube in fluid communication with the inflation lumen of the inflation tube.

12. A balloon apparatus, as in claim 9, further comprising the sleeve secured to the balloon.

13. A balloon apparatus, as in claim 12, the sleeve comprising a disk peripherally secured to the balloon.

14. A balloon apparatus, as in claim 13, further comprising a proximal tube defining a proximal lumen extending between a proximal end and a distal end of the proximal tube, the distal end of the proximal tube secured to a proximal end of the inflation tube with the proximal lumen of the proximal tube in fluid communication with the inflation lumen of the inflation tube.
